(19) United States
(12) Patent Application Publication
(10) Pub. No.: US 2025/0242069 A1
(43) Pub. Date: Jul. 31, 2025

(54) ULTRAVIOLET LIGHT DISINFECTANT DEVICE

(71) Applicant: NORTHLIGHT ELECTRIC COMPANY LIMITED, Hsinchu County (TW)

(72) Inventors: SHEN-HONG KAO, Hsinchu County (TW); I-TING KUO, Hsinchu County (TW)

(21) Appl. No.: 18/422,052

(22) Filed: Jan. 25, 2024

(51) Int. Cl. A61L 2/10; A61L 2/24; A61L 2/26

(52) U.S. Cl. CPC A61L 2/10; A61L 2/24; A61L 2/26; A61L 2202/11; A61L 2202/14; A61L 2202/20

(57) ABSTRACT

An ultraviolet (UV) light disinfectant device includes an ultraviolet (UV) light source, a motion detecting sensor and a controller. The ultraviolet (UV) light disinfectant device is installed on or above an operation interface. The ultraviolet (UV) light source is preferred to be positioned to provide coverage of specific portions of the entire operational surface of the operation interface. The ultraviolet (UV) light source emits UV or UV-C radiation to kill pathogens on the operational surface of the operation interface. The motion detecting sensor is used to detect any motion in the vicinity of the operation interface. When the ultraviolet (UV) light source is in the operation mode to emit UV or UV-C radiation to kill pathogens on the operational surface of the operation interface, if the motion detecting sensor detects any motion of a user in the vicinity of the operation interface, the controller will pause the ultraviolet (UV) light source in the operation mode so as to protect the user.

### 1 TECHNICAL FIELD

The present disclosure relates generally to disinfectant devices. More particularly, the present disclosure relates to ultraviolet (UV) light disinfectant devices.

### 2. DESCRIPTION OF THE RELATED ART

Ensuring appropriate levels of sanitization as means to limit contamination, pathogen transmission, and disease has proven to be a considerable challenge. This has become even more evident as the COVID-19 moved from a localized outbreak to a global pandemic.

It is instructive to examine how the COVID-19 illness spread as a fresh proxy for the overall issue, and to help determine what could have been done to limit this spread. COVID-19, as with many viruses and communicable illnesses, transmits primarily through person-to-person exchange, both by proximity, and from contaminated objects. With people in close proximity to each other droplets from sneezing and coughing can carry the virus from person to person, and commonly touched surfaces if contaminated with virus-containing droplets can similarly cause transmission. In order to reduce transmission, steps must be taken to reduce the amount of airborne and surface live virus that is available for transmission. Frequent hand washing and surface wipe-downs help, but they depend on an individual's vigilance to be effective. The issue of enhanced sanitization has been elevated in our collective consciousness since the onset of the global COVID-19 pandemic, however there are additional uses for the technology than can greatly enhance the levels of sanitization in our daily lives.

Irradiation of surfaces with UV light is a common disinfection method. However, developments in UVC radiation that are effective in disinfecting against target pathogens have enabled new opportunities in sterilization involving human tissue. Public buildings such as malls, office buildings, grocery stores, etc., typically have a number of common public touch points such as door handles, door knobs, elevator buttons, and the like. Most often such common public touch points are touched by a user's bare hands. In some cases, the user may have an illness such as a common cold, influenza, COVID-19 and/or some other illness. In some cases, the user may be asymptomatic but still contagious. When such a user touches a common public touch point, such as a door handle, the user may unintentionally contaminate the touched surface. This contamination may expose subsequent users who touch the same common public touch point to the illness, thereby placing the subsequent users at risk of contracting the illness.

Light switches, elevator call buttons, elevator panel buttons, security keypads, toilet flush switch buttons, ATM panel buttons are a few examples of contact surfaces that are typical vectors for indirect or direct contact transmission of infectious disease. For example, influenza viruses may be transferred via this indirect or direct contact transmission.

Unfortunately, many of the potential contamination transference vectors characteristic of healthcare facilities and public or common areas are not adequately addressed. Electrical or electronic switches (such as light switches, elevator switches, and the like), in common areas such as offices, hotels, hospitals, typically are contaminated. The contamination may be transferred from one user of the switch or button to another at the contact surface of the switch or button when it is used. This contamination transference vector is thought to contribute to the transfer of myriad communicable diseases. Thus, an effective means of keeping the contact surfaces of switches or buttons disinfected is needed.

### SUMMARY

It is an object of the present disclosure to enable the reduction or elimination of transference related contamination from select touch points, including but not limited to electrical switches, and push button switches, such as common light switches, elevator call buttons, elevator panel buttons, keyboards, touch panels and the like, in a manner that is efficient, effective and economical.

The ultraviolet (UV) light disinfectant device mainly includes an ultraviolet (UV) light source, a motion detecting sensor and a controller. The ultraviolet (UV) light disinfectant device in accordance with the present disclosure is installed on or above an operation interface. The ultraviolet (UV) light source is preferred to be positioned to provide coverage of specific portions of the entire operational surface of the operation interface. The ultraviolet (UV) light source emits UV or UV-C radiation to kill pathogens on the operational surface of the operation interface. The motion detecting sensor is used to detect any motion in the vicinity of the operation interface. When the ultraviolet (UV) light source is in the operation mode to emit UV or UV-C radiation to kill pathogens on the operational surface of the operation interface, if the motion detecting sensor detects any motion of a user in the vicinity of the operation interface, the controller will pause the ultraviolet (UV) light source in the operation mode so as to protect the users. Alternatively, when the ultraviolet (UV) light source is in the operation mode to emit UV or UV-C radiation to kill pathogens on the operational surface of the operation interface, if the operation interface is operated by any user, the controller will pause the ultraviolet (UV) light source in the operation mode so as to protect the users.

Objects and advantages of the present disclosure will be set forth in part in the following description, or may be obvious from the description, or may be learned through practice of the present disclosure.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a schematic of an illustrative example of the ultraviolet (UV) light disinfectant device in accordance with the present disclosure.

FIG. 2A is a perspective view of an illustrative example of the ultraviolet (UV) light disinfectant device in accordance with the present disclosure, designed for use with an elevator call buttons in the idle mode.

FIG. 2B is a perspective view of an illustrative example of the ultraviolet (UV) light disinfectant device in accordance with the present disclosure, designed for use with an elevator call buttons in the operation mode.

FIG. 2C is a perspective view of an illustrative example of the ultraviolet (UV) light disinfectant device in accordance with the present disclosure, designed for use with an elevator control panel in the idle mode.

FIG. 2D is a perspective view of an illustrative example of the ultraviolet (UV) light disinfectant device in accordance with the present disclosure, designed for use with an elevator control panel in the operation mode.

FIG. 3A is a perspective view of an illustrative example of the ultraviolet (UV) light disinfectant device in accordance with the present disclosure, designed for use with a keyboard in the idle mode.

FIG. 3B is a perspective view of an illustrative example of the ultraviolet (UV) light disinfectant device in accordance with the present disclosure, designed for use with a keyboard in the operation mode.

FIG. 4A is a perspective view of an illustrative example of the ultraviolet (UV) light disinfectant device in accordance with the present disclosure, designed for use with a touch panel in the idle mode.

FIG. 4B is a perspective view of an illustrative example of the ultraviolet (UV) light disinfectant device in accordance with the present disclosure, designed for use with a touch panel in the operation mode.

FIG. 5A is a perspective view of another illustrative example of the ultraviolet (UV) light disinfectant device in accordance with the present disclosure, designed for use with a touch panel in the idle mode.

FIG. 5B is a perspective view of another illustrative example of the ultraviolet (UV) light disinfectant device in accordance with the present disclosure, designed for use with a touch panel in the operation mode.

### DETAILED DESCRIPTION OF THE PREFERRED EMBODIMENT

The following disclosure provides many different embodiments, or examples, for implementing different features of the invention. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Further, spatial relative terms, such as “beneath,” “below,” “lower,” “above,” “upper” and the like, may be used herein for ease of description to describe one element or feature's relationship to another element(s) or feature(s) as illustrated in the figures. The spatial relative terms are intended to encompass different orientations of the device in use or operation in addition to the orientation depicted in the figures.

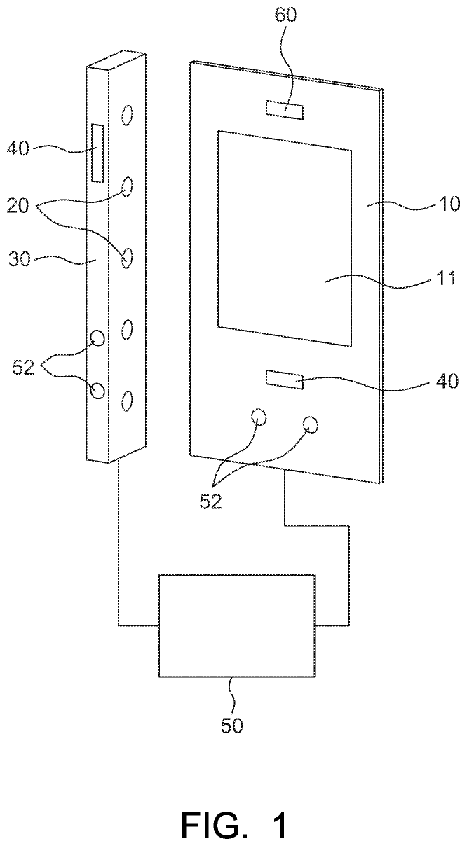

Referring to FIG. 1, it shows a schematic of an illustrative example of the ultraviolet (UV) light disinfectant device in accordance with the present disclosure. The ultraviolet (UV) light disinfectant device mainly an ultraviolet (UV) light source (20) mounted in a frame (30), a motion detecting sensor (40) and a controller (50). The ultraviolet (UV) light disinfectant device in accordance with the present disclosure is installed on or above an operation interface (10), such as light switch, elevator call button, or elevator control panel, security keypad, ATM panel button and touch panel of electronic device.

The ultraviolet (UV) light source (20) mounted in the frame (30) and is preferred to be positioned to provide coverage of specific portions of the entire operational surface (11) of the operation interface (10). The ultraviolet (UV) light source (20) mainly emits UV or UV-C radiation to kill pathogens on the operational surface (11) of the operation interface (10). Other effective UV-C generators or bulbs may be used. As will be understood by one skilled in the art, the UV or UV-C generators or bulbs may be one or more arrays of light emitting diodes (LEDs) that emit UV or UV-C radiation.

The motion detecting sensor (40) is used to detect any motion in the vicinity of the operation interface (10). As will be understood by one skilled in the art, passive infrared (PIR), microwave, and dual type motion detecting sensors that detect any motion in the vicinity of the operation interface (10) can be implemented in the ultraviolet (UV) light disinfectant device in accordance with the present disclosure. When the ultraviolet (UV) light source (20) is in the operation mode to emit UV or UV-C radiation to kill pathogens on the operational surface (11) of the operation interface (10), if the motion detecting sensor (40) detects any motion of a user in the vicinity of the operation interface (10), the controller (50) will pause the ultraviolet (UV) light source (20) in the operation mode so as to protect the users. Alternatively, when the ultraviolet (UV) light source (20) is in the operation mode to emit UV or UV-C radiation to kill pathogens on the operational surface (11) of the operation interface (10), if the operation interface (10) is operated by any user, the controller (50) will pause the ultraviolet (UV) light source (20) in the operation mode so as to protect the users.

After the operation interface (10) is used by the user, the controller (50) outputs an operation mode signal to the ultraviolet (UV) light source (20) for emitting UV or UV-C radiation to kill pathogens on the operational surface (11) of the operation interface (10) for a predetermined period preset in the controller (50) according to the power of the ultraviolet (UV) light source (20) and the disinfectant area size. In the meanwhile, the indictor light (52) shows in the operation mode, for example, in orange color, in response to the operation mode signal output from the controller (50). According to some embodiments of the present disclosure, the frame (30) is positioned in an idle position when the ultraviolet (UV) light source (20) is not in the operation mode, and is moved to an operation position such that the Ultraviolet (UV) light source (20) mounted in the frame (30) and is positioned to provide coverage of specific portions of the entire operational surface (11) of the operation interface (10) when the ultraviolet (UV) light source (20) is in the operation mode. The movement of the frame (30) between the idle position and the operation position is controlled manually by the user or controlled by the operation mode signal output from the controller (50). After the ultraviolet (UV) light source (20) emitting UV or UV-C radiation for the predetermined period, the ultraviolet (UV) light disinfectant device completes the entire procedure to kill pathogens on the operational surface (11) of the operation interface (10)

During the ultraviolet (UV) light source (20) in the operation mode for emitting UV or UV-C radiation to kill pathogens on the operational surface (11) of the operation interface (10), if the motion detecting sensor (40) detects any motion of a user in the vicinity of the operation interface (10), the controller (50) will immediately output a pause signal to set the ultraviolet (UV) light source (20) in pause mode to pause the ultraviolet (UV) light source (20) to emit UV or UV-C radiation for a predetermined time, for example 30 seconds, to prevent the user from harming by the UV or UV-C radiation so as to protect the users. In the meanwhile, the indictor light (52) shows in the warming mode, for example, in red color, in response to the pause mode signal output from the controller (50). According to some embodiments of the present disclosure, the frame (30) is moved from the operation position back to the idle position in response to the pause signal output from the controller (50).

Alternatively, when the ultraviolet (UV) light source (20) is in the operation mode to emit UV or UV-C radiation to kill pathogens on the operational surface (11) of the operation interface (10), if the operation interface (10) is operated by any user, the controller (50) will immediately output a pause signal to set the ultraviolet (UV) light source (20) in pause mode to pause the ultraviolet (UV) light source (20) to emit UV or UV-C radiation for a predetermined time, for example 30 seconds, to prevent the user from harming by the UV or UV-C radiation so as to protect the users. In the meanwhile, the indictor light (52) shows in the warming mode, for example, in red color, in response to the pause mode signal output from the controller (50). According to some embodiments of the present disclosure, the frame (30) is moved from the operation position back to the idle position in response to the pause signal output from the controller (50).

After the ultraviolet (UV) light source (20) has been in the pause mode for the predetermined time, for example 30 seconds, and the motion detecting sensor (40) does not detect any motion of a user in the vicinity of the operation interface (10) or the operation interface (10) is operated, the controller (50) outputs another operation mode signal to the ultraviolet (UV) light source (20), thereby resuming the ultraviolet (UV) light source (20) for emitting UV or UV-C radiation so as to complete the entire procedure to kill pathogens on the operational surface (11) of the operation interface (10).

Alternatively, the ultraviolet (UV) light disinfectant device in accordance with the present disclosure further comprises a test button (60) on the operation interface (10). When testing, the engineer can use the test button (60) to activate the ultraviolet (UV) light source (20) to emit UV or UV-C radiation to kill pathogens on the operational surface (11) of the operation interface (10) to ensure the ultraviolet (UV) light disinfectant device functional in all aspects.

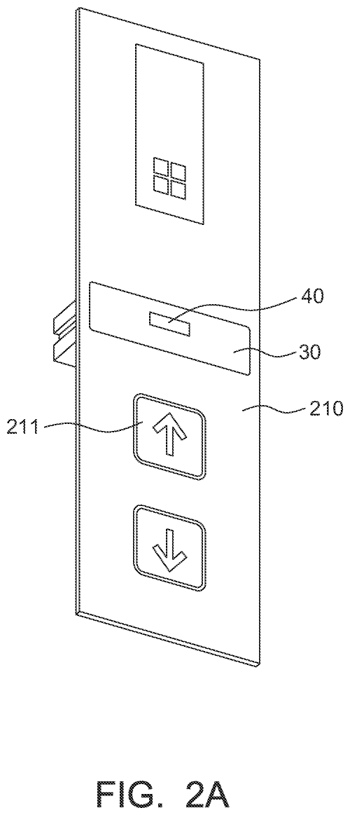

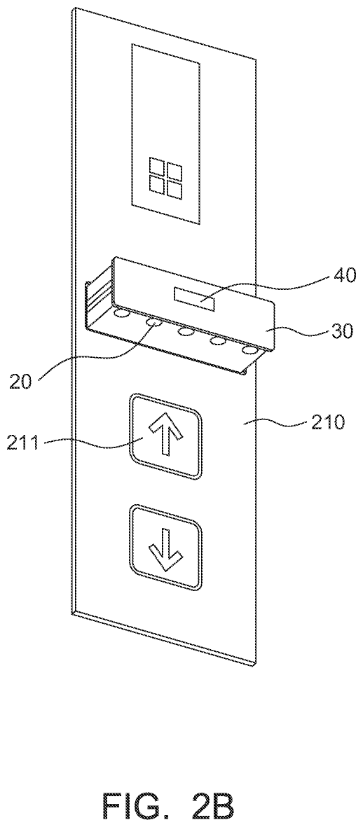

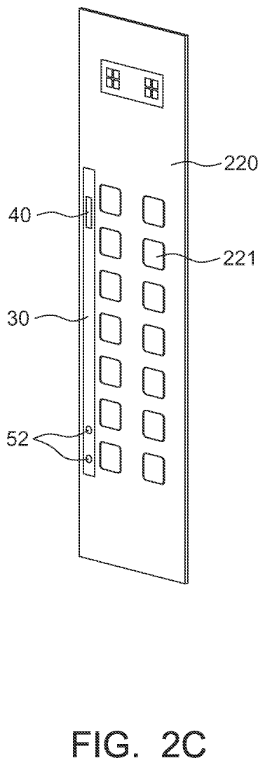

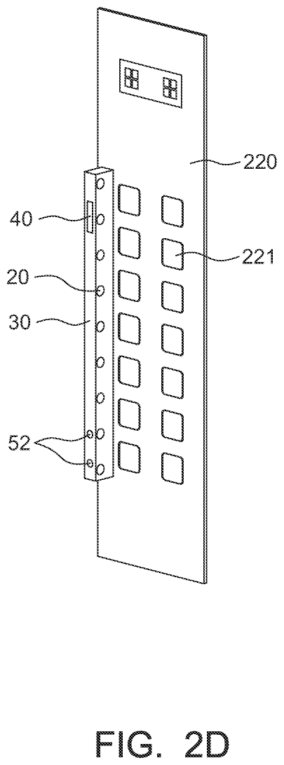

FIG. 2A, FIG. 2B, FIG. 2C and FIG. 2D are perspective views of an illustrative example of the ultraviolet (UV) light disinfectant device in accordance with the present disclosure, designed for use with an elevator call button and an elevator control panel respectively in the idle mode and in the operation mode. In these embodiments, the elevator call button and the elevator control panel are the operation interface (10) as shown in FIG. 1. In these embodiments in accordance with the present disclosure, as shown in FIG. 2A, when the frame (30) is in the idle position, the frame (30) together with the ultraviolet (UV) light source (20) is positioned in the same plane with the elevator call button (210) or the elevator control panel (220). When the ultraviolet (UV) light source (20) is in the operation mode, the frame (30) is moved out to an operation position in response to the to the operation mode signal output from the controller (50) such that the ultraviolet (UV) light source (20) mounted in the frame (30) is positioned to provide coverage of specific portions of the entire push bottoms (211) of the elevator call button (210) or the push bottoms (221) of the control panel (220).

As shown in FIG. 2B, the ultraviolet (UV) light source (20) mounted in the frame (30) of an elongated housing and the frame (30) is mounted on and positioned next to the entire of the elevator call button (210) or the entire elevator control panel (220) to provide coverage of the entire push bottoms (211, 321). The indictor light (52) is provided on the the elevator call button (210) or the elevator control panel (220), and is preferably provided at the outer surface of the frame (30). The motion detecting sensor (40) is either positioned on or inside the frame (30) together with the ultraviolet (UV) light source (20) or on the elevator call button (210) or the elevator control panel (220) outside the frame (30) as long as the motion detecting sensor (40) can detect any motion in the vicinity of the elevator call button (210) or the elevator control panel (220) within the coverage of the UV or UV-C radiation emitted from the ultraviolet (UV) light source (20).

After the elevator call button (210) or the elevator control panel (220) is pressed by the user, the controller (50) outputs an operation mode signal to the ultraviolet (UV) light source (20) for emitting UV or UV-C radiation to kill pathogens on push bottoms (211, 221) of the elevator call button (210) or the elevator control panel (320) for a predetermined period preset in the controller (50). In the meanwhile, the indictor light (52) shows in the operation mode, for example, in orange color, in response to the operation mode signal output from the controller (50). After the ultraviolet (UV) light source (20) emitting UV or UV-C radiation for the predetermined period, the ultraviolet (UV) light disinfectant device completes the entire procedure to kill pathogens on the push bottoms (211, 221) of the elevator call button (210) or the elevator control panel (220)

During the ultraviolet (UV) light source (20) in the operation mode for emitting UV or UV-C radiation, if the motion detecting sensor (40) detects any motion of a user in the vicinity of the elevator call button (210) or the elevator control panel (220), the controller (50) will immediately output a pause signal to pause the ultraviolet (UV) light source (20) to emit UV or UV-C radiation for a predetermined time, for example 30 seconds, in the pause mode to prevent the user from harming by the UV or UV-C radiation. In the meanwhile, the indictor light (52) shows in the warming mode, for example, in red color, in response to the pause mode signal output from the controller (50). After the ultraviolet (UV) light source (20) has been in the pause mode for the predetermined time, for example 30 seconds, and the motion detecting sensor (40) does not detect any motion of a user in the vicinity of the elevator call button (210) or the elevator control panel (220), the controller (50) outputs another operation mode signal to the ultraviolet (UV) light source (20), thereby resuming the ultraviolet (UV) light source (20) for emitting UV or UV-C radiation so as to complete the entire procedure to kill pathogens on the push bottoms (211, 221) of the elevator call button (210) or the elevator control panel (220). Alternatively, when the ultraviolet (UV) light source (20) is in the operation mode to emit UV or UV-C radiation to kill pathogens on the entire push bottoms (211, 221) of the elevator call button (210) or the elevator control panel (220), if the elevator call button (210) or the elevator control panel (220) is operated by any user, the controller (50) will pause the ultraviolet (UV) light source (20) in the operation mode so as to protect the users. After the ultraviolet (UV) light source (20) has been in the pause mode for the predetermined time, for example 30 seconds, and the elevator call button (210) or the elevator control panel (220) is not operated by any user, the controller (50) outputs another operation mode signal to the ultraviolet (UV) light source (20), thereby resuming the ultraviolet (UV) light source (20) for emitting UV or UV-C radiation so as to complete the entire procedure to kill pathogens on the push bottoms (211, 221) of the elevator call button (210) or the elevator control panel (220).

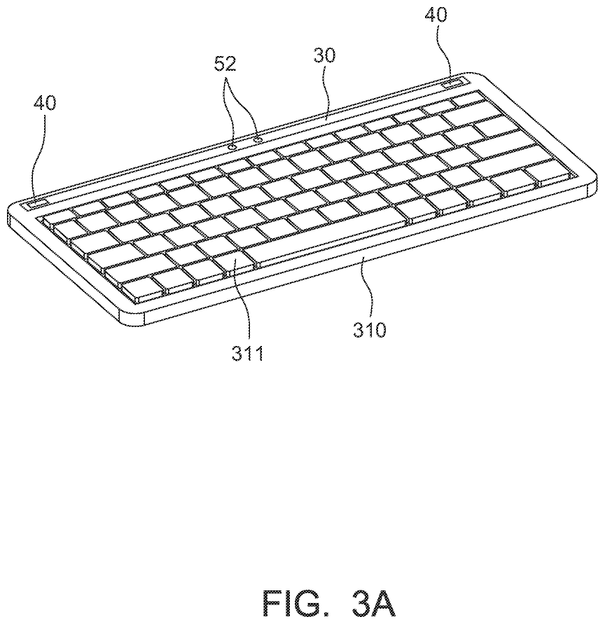

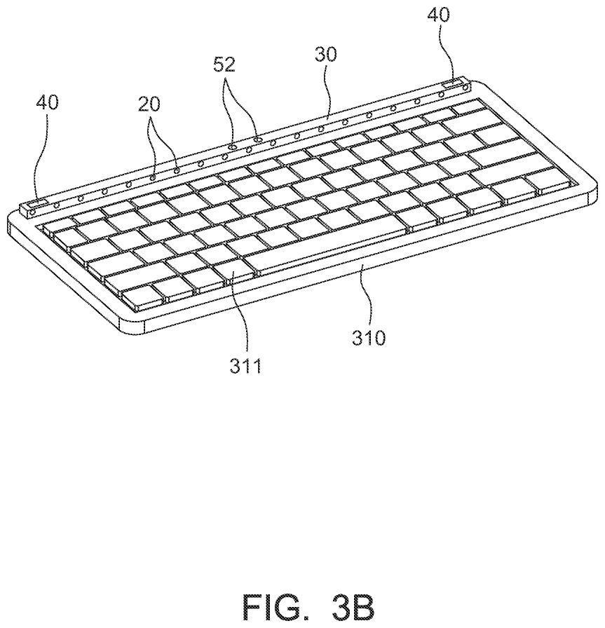

FIG. 3A and FIG. 3B are perspective views of an illustrative example of the ultraviolet (UV) light disinfectant device in accordance with the present disclosure, designed for use with a keyboard respectively in the idle mode and in the operation mode. In this embodiment, the keyboard is the operation interface (10) as shown in FIG. 1. In this embodiment in accordance with the present disclosure, as shown in FIG. 3A, when the frame (30) is in the idle position, the frame (30) together with the ultraviolet (UV) light source (20) is positioned in the same plane with the keyboard (310). When the ultraviolet (UV) light source (20) is in the operation mode, the frame (30) is moved up to an operation position in response to the operation mode signal output from the controller (50) such that the ultraviolet (UV) light source (20) mounted in the frame (30) is positioned to provide coverage of specific portions of the entire push bottoms (311) of the keyboard (310). The ultraviolet (UV) light source (20) mounted in the frame (30) of an elongated housing and the frame (30) is mounted on the upper end of the keyboard (310) and positioned next to the entire keyboard to provide coverage of the entire push bottoms (311) of the keyboard (310). The indictor light (52) is preferably provided at the outer surface of the frame (30). The motion detecting sensor (40) is either positioned inside or on the frame (30) together with the ultraviolet (UV) light source (20) or on the keyboard (310) outside the frame (30) as long as the motion detecting sensor (40) can detect any motion in the vicinity of the keyboard (310) within the coverage of the UV or UV-C radiation emitted from the ultraviolet (UV) light source (20).

After the keyboard (310) is used by the user, the controller (50) outputs an operation mode signal to the ultraviolet (UV) light source (20) for emitting UV or UV-C radiation to kill pathogens on push bottoms (311) of the keyboard (310) for a predetermined period preset in the controller (50). In the meanwhile, the indictor light (52) shows in the operation mode, for example, in orange color, in response to the operation mode signal output from the controller (50). After the ultraviolet (UV) light source (20) emitting UV or UV-C radiation for the predetermined period, the ultraviolet (UV) light disinfectant device completes the entire procedure to kill pathogens on the push bottoms (311) of the keyboard (310)

During the ultraviolet (UV) light source (20) in the operation mode for emitting UV or UV-C radiation, if the motion detecting sensor (40) detects any motion of a user in the vicinity of the keyboard (310), the controller (50) will immediately output a pause signal to pause the ultraviolet (UV) light source (20) to emit UV or UV-C radiation for a predetermined time, for example 30 seconds, in the pause mode to prevent the user from harming by the UV or UV-C radiation. In the meanwhile, the indictor light (52) shows in the warming mode, for example, in red color, in response to the pause mode signal output from the controller (50). After the ultraviolet (UV) light source (20) has been in the pause mode for the predetermined time, for example 30 seconds, and the motion detecting sensor (40) does not detect any motion of a user in the vicinity of the keyboard (310), the controller (50) outputs another operation mode signal to the ultraviolet (UV) light source (20), thereby resuming the ultraviolet (UV) light source (20) for emitting UV or UV-C radiation so as to complete the entire procedure to kill pathogens on the push bottoms (311) of the keyboard (310).

In this embodiment in accordance with the present disclosure, as shown in FIG. 3A, when the frame (30) is in the idle position, the frame (30) together with the ultraviolet (UV) light source (20) is positioned in the same plane with the push bottoms (311) of the keyboard (310). When the ultraviolet (UV) light source (20) is in the operation mode, the frame (30) is moved up to an operation position in response to the to the operation mode signal output from the controller (50) such that the ultraviolet (UV) light source (20) mounted in the frame (30) is positioned to provide coverage of specific portions of the entire push bottoms (311) of the keyboard (310).

Alternatively, when the ultraviolet (UV) light source (20) is in the operation mode to emit UV or UV-C radiation to kill pathogens on the entire push bottoms (311) of the keyboard (310), if the keyboard (310) is operated by any user, the controller (50) will pause the ultraviolet (UV) light source (20) in the operation mode so as to protect the users. After the ultraviolet (UV) light source (20) has been in the pause mode for the predetermined time, for example 30 seconds, and the keyboard (310) is not operated by any user, the controller (50) outputs another operation mode signal to the ultraviolet (UV) light source (20), thereby resuming the ultraviolet (UV) light source (20) for emitting UV or UV-C radiation so as to complete the entire procedure to kill pathogens on the push bottoms (311) of the keyboard (310).

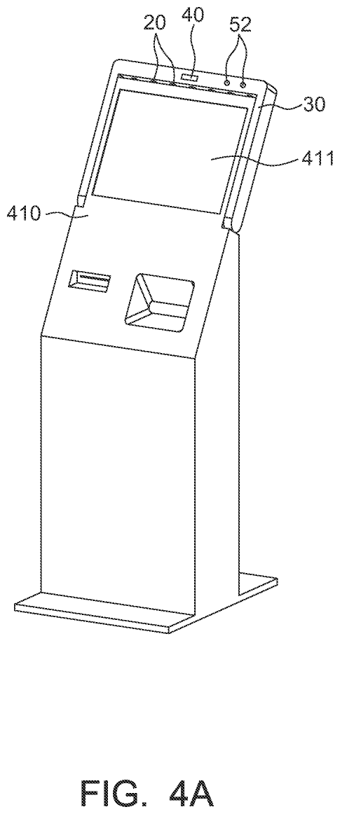

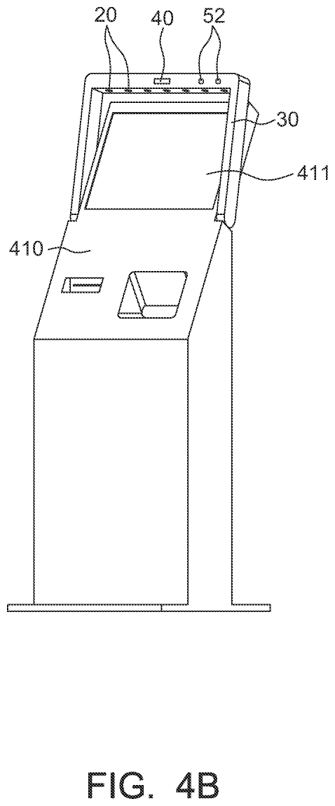

FIG. 4A and FIG. 4B are perspective views of an illustrative example of the ultraviolet (UV) light disinfectant device in accordance with the present disclosure, designed for use with a touch panel respectively in the idle mode and in the operation mode. In this embodiment, the touch panel is the operation interface (10) as shown in FIG. 1. In this embodiment in accordance with the present disclosure, as shown in FIG. 4A, when the frame (30) is in the idle position, the frame (30) together with the ultraviolet (UV) light source (20) is positioned in the same plane with the touch panel (510). When the ultraviolet (UV) light source (20) is in the operation mode, the frame (30) is moved up to an operation position in response to the operation mode signal output from the controller (50) such that the ultraviolet (UV) light source (20) mounted in the frame (30) is positioned to provide coverage of specific portions of the entire touch panel (410). The ultraviolet (UV) light source (20) mounted in the frame (30) of a U-shape housing and the two ends of the U-shape frame (30) is pivotally mounted on both sides of the touch panel (410). The indictor light (52) is preferably provided at the outer surface of the frame (30) or on the the touch panel (410). The motion detecting sensor (40) is either positioned inside or on the frame (30) together with the ultraviolet (UV) light source (20) or on the touch panel (410) outside the frame (30) as long as the motion detecting sensor (40) can detect any motion in the vicinity of the touch panel (410) within the coverage of the UV or UV-C radiation emitted from the ultraviolet (UV) light source (20).

After the touch panel (410) is used by the user, the controller (50) outputs an operation mode signal to the ultraviolet (UV) light source (20) for emitting UV or UV-C radiation to kill pathogens on the touching surface (411) of the touch panel (410) for a predetermined period preset in the controller (50). In the meanwhile, the indictor light (52) shows in the operation mode, for example, in orange color, in response to the operation mode signal output from the controller (50). After the ultraviolet (UV) light source (20) emitting UV or UV-C radiation for the predetermined period, the ultraviolet (UV) light disinfectant device completes the entire procedure to kill pathogens on the touch panel (410)

During the ultraviolet (UV) light source (20) in the operation mode for emitting UV or UV-C radiation, if the motion detecting sensor (40) detects any motion of a user in the vicinity of the touch panel (410), the controller (50) will immediately output a pause signal to pause the ultraviolet (UV) light source (20) to emit UV or UV-C radiation for a predetermined time, for example 30 seconds, in the pause mode to prevent the user from harming by the UV or UV-C radiation. In the meanwhile, the indictor light (52) shows in the warming mode, for example, in red color, in response to the pause mode signal output from the controller (50). After the ultraviolet (UV) light source (20) has been in the pause mode for the predetermined time, for example 30 seconds, and the motion detecting sensor (40) does not detect any motion of a user in the vicinity of the touch panel (410), the controller (50) outputs another operation mode signal to the ultraviolet (UV) light source (20), thereby resuming the ultraviolet (UV) light source (20) for emitting UV or UV-C radiation so as to complete the entire procedure to kill pathogens on the touching surface (411) of the touch panel (410).

Alternatively, when the ultraviolet (UV) light source (20) is in the operation mode to emit UV or UV-C radiation to kill pathogens on the the touching surface (411) of the touch panel (410), if the touch panel (510) is operated by any user, the controller (50) will pause the ultraviolet (UV) light source (20) in the operation mode so as to protect the users. After the ultraviolet (UV) light source (20) has been in the pause mode for the predetermined time, for example 30 seconds, and the touch panel (510) is not operated by any user, the controller (50) outputs another operation mode signal to the ultraviolet (UV) light source (20), thereby resuming the ultraviolet (UV) light source (20) for emitting UV or UV-C radiation so as to complete the entire procedure to kill pathogens on the touching surface (411) of the touch panel (410).

In this embodiment in accordance with the present disclosure, when the frame (30) is in the idle position, the frame (30) together with the ultraviolet (UV) light source (20) is positioned in the same plane with the touch panel (410). When the ultraviolet (UV) light source (20) is in the operation mode, the frame (30) is moved up to an operation position in response to the to the operation mode signal output from the controller (50) such that the Ultraviolet (UV) light source (20) mounted in the frame (30) and is positioned to provide coverage of specific portions of the entire touching surface (411) of the touch panel (410).

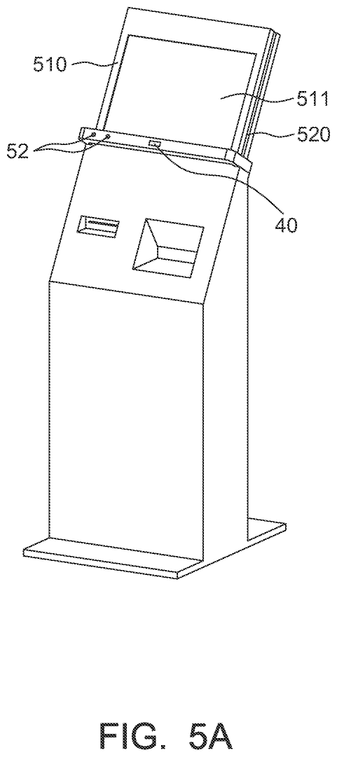

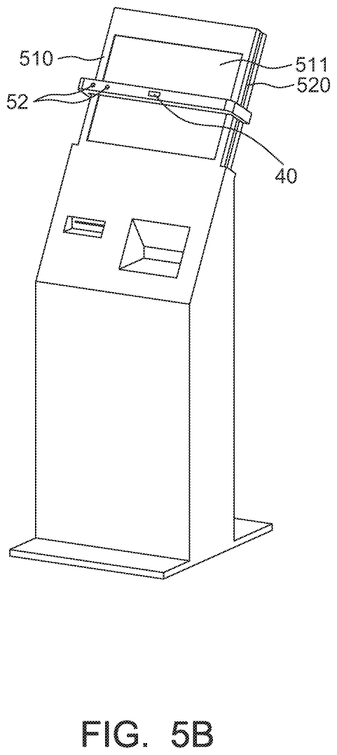

FIG. 5A and FIG. 5B are perspective views of an illustrative example of the ultraviolet (UV) light disinfectant device in accordance with the present disclosure, designed for use with a touch panel respectively in the idle mode and in the operation mode. In this embodiment in accordance with the present disclosure, as shown in FIG. 5A, when the frame (30) is in the idle position, the frame (30) together with the ultraviolet (UV) light source (20) is positioned at the lower end of the touch panel (510). The frame (30) is slidable within the two slots (520) positioned at both sides of the touch panel (510). When the ultraviolet (UV) light source (20) is in the operation mode, the frame (30) is moved from the lower of the touch panel (510) towards the upper end of the touch panel (510) in response to the operation mode signal output from the controller (50) such that the ultraviolet (UV) light source (20) mounted in the frame (30) is positioned to provide coverage of the entire touch panel (510). Alternatively, when the frame (30) is in the idle position, the frame (30) together with the ultraviolet (UV) light source (20) is positioned at the upper end of the touch panel (510). The frame (30) is slidable within the two slots (520) positioned at both sides of the touch panel (510). When the ultraviolet (UV) light source (20) is in the operation mode, the frame (30) is moved from the upper end of the touch panel (510) towards the lower end of the touch panel (510) in response to the operation mode signal output from the controller (50) such that the ultraviolet (UV) light source (20) mounted in the frame (30) is positioned to provide coverage of the entire touch panel (510). The indictor light (52) is preferably provided at the outer surface of the frame (30) or on the the touch panel (510). The motion detecting sensor (40) is either positioned inside or on the frame (30) together with the ultraviolet (UV) light source (20) or on the touch panel (610) outside the frame (30) as long as the motion detecting sensor (40) can detect any motion in the vicinity of the touch panel (510) within the coverage of the UV or UV-C radiation emitted from the ultraviolet (UV) light source (20).

After the touch panel (510) is used by the user, the controller (50) outputs an operation mode signal to the ultraviolet (UV) light source (20) for emitting UV or UV-C radiation to kill pathogens on the touching surface (511) of the touch panel (510) for a predetermined period preset in the controller (50). In the meanwhile, the indictor light (52) shows in the operation mode, for example, in orange color, in response to the operation mode signal output from the controller (50). After the ultraviolet (UV) light source (20) emitting UV or UV-C radiation for the predetermined period, the ultraviolet (UV) light disinfectant device completes the entire procedure to kill pathogens on the touch panel (510)

During the ultraviolet (UV) light source (20) in the operation mode for emitting UV or UV-C radiation, if the motion detecting sensor (40) detects any motion of a user in the vicinity of the touch panel (510), the controller (50) will immediately output a pause signal to pause the ultraviolet (UV) light source (20) to emit UV or UV-C radiation for a predetermined time, for example 30 seconds, in the pause mode to prevent the user from harming by the UV or UV-C radiation. In the meanwhile, the indictor light (52) shows in the warming mode, for example, in red color, in response to the pause mode signal output from the controller (50). After the ultraviolet (UV) light source (20) has been in the pause mode for the predetermined time, for example 30 seconds, and the motion detecting sensor (40) does not detect any motion of a user in the vicinity of the touch panel (510), the controller (50) outputs another operation mode signal to the ultraviolet (UV) light source (20), thereby resuming the ultraviolet (UV) light source (20) for emitting UV or UV-C radiation so as to complete the entire procedure to kill pathogens on the touching surface (511) of the touch panel (510).

Alternatively, when the ultraviolet (UV) light source (20) is in the operation mode to emit UV or UV-C radiation to kill pathogens on the touching surface (511) of the touch panel (510), if the touch panel (610) is operated by any user, the controller (50) will pause the ultraviolet (UV) light source (20) in the operation mode so as to protect the users. After the ultraviolet (UV) light source (20) has been in the pause mode for the predetermined time, for example 30 seconds, and the touch panel (510) is not operated by any user, the controller (50) outputs another operation mode signal to frame (30) and the ultraviolet (UV) light source (20), thereby resuming frame (30) continuing moving and the ultraviolet (UV) light source (20) for emitting UV or UV-C radiation so as to complete the entire procedure to kill pathogens on the touching surface (511) of the touch panel (510).

It will become apparent to those people skilled in the art that various modifications and variations can be made to the structure of the disclosure without departing from the scope or spirit of the disclosure. In view of the foregoing description, it is intended that all the modifications and variation fall within the scope of the following appended claims and their equivalents.

What is claimed is:

1. A disinfectant device, comprising:
a ultraviolet (UV) light source configured to emit UV or UV-C radiation on an operation interface;
a motion detecting sensor configured to detect any motion in the vicinity of the operation interface;
a controller configured to
after the operation interface is used by a user, output an operation mode signal to set the ultraviolet (UV) light source in an operation mode for emitting the UV or UV-C radiation to kill pathogens on the operation interface for a first predetermined period preset in the controller according to the disinfectant area size of the operation interface;
when the motion detecting sensor detects any motion in the vicinity of the operation interface during the ultraviolet (UV) light source in the operation mode, output a pause signal to set the Ultraviolet (UV) light source in a pause mode to pause the Ultraviolet (UV) light to emit UV or UV-C radiation for a second predetermined time to prevent the user from harming by the UV or UV-C radiation; and
after the ultraviolet (UV) light source has been in the pause mode for the second predetermined time and the motion detecting sensor does not detect any motion in the vicinity of the operation interface, output another operation mode signal to the ultraviolet (UV) light source, thereby resuming the ultraviolet (UV) light source for emitting UV or UV-C radiation so as to complete the entire procedure to kill pathogens on the operation interface.

2. The disinfectant device as claimed in claim 1, further comprising an indictor light configured to show in the operation mode in response to the operation mode signal output from the controller, and in the warming mode in response to the pause mode signal output from the controller.

3. The disinfectant device as claimed in claim 1, further comprising a frame for accommodating the ultraviolet (UV) light source therein, wherein the ultraviolet (UV) light source is positioned to provide coverage of specific portions of the entire of the operation interface.

4. The disinfectant device as claimed in claim 1, wherein the frame is positioned in an idle position when the ultraviolet (UV) light source is not in the operation mode, and is moved to an operation position such that the ultraviolet (UV) light source mounted in the frame is positioned to provide coverage of specific portions of the entire the operation interface when the ultraviolet (UV) light source is in the operation mode.

5. The disinfectant device as claimed in claim 1, wherein the frame is moved between the idle position and the operation position manually by the user.

6. The disinfectant device as claimed in claim 1, wherein the frame is moved from the idle position to the operation position in accordance with the by the operation mode signal output from the controller.

7. The disinfectant device as claimed in claim 3, wherein the operation interface is an elevator control panel, and the frame is an elongated housing mounted on and positioned next to the entire elevator control panel.

8. The disinfectant device as claimed in claim 3, wherein the operation interface is a keyboard, and the frame is an elongated housing mounted on the upper end of the keyboard and positioned next to the entire keyboard to provide coverage of the entire keyboard.

9. The disinfectant device as claimed in claim 8, wherein when ultraviolet (UV) light source is not in the operation mode, the frame is in an idle position where the frame together with the ultraviolet (UV) light source is positioned in the same plane with the keyboard, and when the ultraviolet (UV) light source is in the operation mode, the frame is moved up to an operation position in response to the to the operation mode signal output from the controller such that the ultraviolet (UV) light source in the frame is positioned to provide coverage of specific portions of the entire keyboard.

10. The disinfectant device as claimed in claim 3, wherein the operation interface is a touch panel, and the frame is a U-shape housing and two ends of the U-shape frame is pivotally mounted on both sides of the touch panel.

11. The disinfectant device as claimed in claim 10, wherein when ultraviolet (UV) light source is not in the operation mode, the frame is in an idle position where the frame together with the ultraviolet (UV) light source is positioned in the same plane with the touch panel, and when the ultraviolet (UV) light source is in the operation mode, the frame is moved up to an operation position where the ultraviolet (UV) light source in the frame is positioned to provide coverage of specific portions of the entire touch panel.

12. A disinfectant device, comprising:
a ultraviolet (UV) light source configured to emit UV or UV-C radiation on an operation interface;
a controller configured to
after the operation interface is used by a user, output an operation mode signal to set the ultraviolet (UV) light source in an operation mode for emitting the UV or UV-C radiation to kill pathogens on the operation interface for a first predetermined period preset in the controller according to the disinfectant area size of the operation interface;
when the operation interface is used by any users during the ultraviolet (UV) light source in the operation mode, output a pause signal to set the Ultraviolet (UV) light source in a pause mode to pause the Ultraviolet (UV) light to emit UV or UV-C radiation for a second predetermined time to prevent the user from harming by the UV or UV-C radiation; and
after the ultraviolet (UV) light source has been in the pause mode for the second predetermined time and the operation interface is not used, output another operation mode signal to the ultraviolet (UV) light source, thereby resuming the ultraviolet (UV) light source for emitting UV or UV-C radiation so as to complete the entire procedure to kill pathogens on the operation interface.

13. The disinfectant device as claimed in claim 12, further comprising an indictor light configured to show in the operation mode in response to the operation mode signal output from the controller, and in the warming mode in response to the pause mode signal output from the controller.

14. The disinfectant device as claimed in claim 12, further comprising a frame for accommodating the ultraviolet (UV) light source therein, wherein the ultraviolet (UV) light source is positioned to provide coverage of specific portions of the entire of the operation interface.

15. The disinfectant device as claimed in claim 12, wherein the frame is positioned in an idle position when the ultraviolet (UV) light source is not in the operation mode, and is moved to an operation position such that the ultraviolet (UV) light source mounted in the frame is positioned to provide coverage of specific portions of the entire the operation interface when the ultraviolet (UV) light source is in the operation mode.

16. The disinfectant device as claimed in claim 12, wherein the frame is moved between the idle position and the operation position manually by the user.

17. The disinfectant device as claimed in claim 12, wherein the frame is moved from the idle position to the operation position in accordance with the by the operation mode signal output from the controller.

18. The disinfectant device as claimed in claim 14, wherein the frame is slidable within the two slots positioned at both sides of the operation interface, and when the ultraviolet (UV) light source is in the operation mode, the frame is moved from one end of the operation interface towards the other end of the operation interface in response to the operation mode signal output from the controller.

19. The disinfectant device as claimed in claim 14, wherein the operation interface is a keyboard, and the frame is an elongated housing mounted on the upper end of the keyboard and positioned next to the entire keyboard to provide coverage of the entire keyboard.

20. The disinfectant device as claimed in claim 14, wherein when ultraviolet (UV) light source is not in the operation mode, the frame is in an idle position where the frame together with the ultraviolet (UV) light source is positioned in the same plane with the operation interface, and when the ultraviolet (UV) light source is in the operation mode, the frame is moved up to an operation position in response to the to the operation mode signal output from the controller such that the ultraviolet (UV) light source in the frame is positioned to provide coverage of specific portions of the operation interface.
